(19) United States
(12) Patent Application Publication
(10) Pub. No.: US 2014/0287474 A1
(43) Pub. Date: Sep. 25, 2014

(54) METHOD OF TREATING ORGANIC MATERIAL TO PRODUCE METHANE GAS

(71) Applicants: Goran Karlsson, Helsingborg (SE); Anders Carlius, Lund (SE)

(72) Inventors: Goran Karlsson, Helsingborg (SE); Anders Carlius, Lund (SE)

(73) Assignee: REACFUEL AB, Lund (SE)

(21) Appl. No.: 14/111,635

(22) PCT Filed: Apr. 13, 2012
(86) PCT No.: PCT/SE12/50406

§ 371 Date: May 16, 2014

Related U.S. Application Data
(60) Provisional application No. 61/475,992, filed on Apr. 15, 2011.

(30) Foreign Application Priority Data
Apr. 15, 2011 (SE) 1150332-3

(51) Int. Cl. C12P 5/02

(52) U.S. Cl. CPC C12P 5/023
USPC 435/167

(57) ABSTRACT

The present invention relates to a method of treating organic materials to produce methane gas, comprising the steps of: a) subjecting an organic feedstock comprising organic materials to a liquefaction process at subcritical conditions in at least one reaction stage, to obtain a mixture containing low molecular weight materials and optionally lignins; b) subjecting the obtained mixture containing low molecular weight materials and optionally lignins, to a methane fermentation process; wherein said organic feedstock comprises liquid water and/or is combined with liquid water before and/or during said liquefaction, and the subcritical conditions for said at least one or more reaction stage(s) in a) is a temperature of 280-374° C. during a reaction time of less than 1 minute, wherein if more than one reaction stage is used in step a) the obtained mixture after each reaction stage is subjected to a separation of the produced low molecular weight materials from the remaining solid materials of the treated feedstock.

### FIELD OF THE INVENTION

The present invention relates to a process of degradation of organic materials and production of methane gas.

### TECHNICAL BACKGROUND

Different processes for degrading and converting organic material into value-adding compounds are known. Degradation of organic matter in sub- or super-critical conditions is known. Anaerobic fermentation, digestion, of organic materials, such as biomass and wastes, is an increasingly common method of producing energy in the form of biogas comprising primarily methane and carbon dioxide, which could be upgraded to methane. Also, different pretreatments before an anaerobic fermentation are known, e.g. grinding, use of ultrasound, steam explosion, use of N-methylmorpholine-N-oxide (NMMO), and treatment in sub- or super-critical conditions.

Anaerobic digestion of wastewater sludge is a common method of reducing the sludge volumes and at the same time obtaining a value-adding product, an energy source. Mesophilic digestion occurs usually at about 35° C. for 20-25 days. Thermophilic digestion occurs at about 50° C. with a shorter retention time. The mesophilic digestion is more commonly used but thermophilic digestion of sludge is increasing with as the demand for and usage of biogas increases. Another factor is a need for sanitizing sludge in order to qualify the sludge for application onto farmlands. For such an application thermophilic digestion may be interesting. The most commonly used substrates are corn and biomass.

Methane fermentation is performed under anaerobic conditions with influence of bacteria. Suitable start materials are e.g. agriculture waste or waste from people, organic materials, which preferably are not containing large amounts of lignin. A common feature for these substrates is that the retention time of the digestion can be very long, sometimes up to 6 months.

JP 2001-262162 discloses a method for producing fuel from biomass. The method includes providing a biomass, degrading the raw materials, which have been made into a slurry with water, in subcritical or supercritical state to reduce the molecular weights and thereafter carry out methane fermentation on the liquid obtained after the degradation by usage of bacteria. The first degradation process at subcritical or supercritical state is performed at a temperature of about 200-500° C., a pressure of about 10-30 MPa and during 1 minute to 10 hours. The following fermentation is performed for 10-100 hours.

EP 1 561 730 discloses a method for producing methane gas. The method includes treating organic wastes with at least one of supercritical water and subcritical water to convert the organic wastes into low molecular weight substances, and then subject the liquid low molecular weight substances to methane fermentation. In subcritical treatment the temperature is about 440-553 K and at a pressure of about 0.8-6.4 MPa and during 1-20 minutes. The methane fermentation is carried out under conventional conditions at a temperature of about 37-55° C. for about 5-48 hours.

When degradation of organic materials is performed, often either the reaction is not driven far enough so that only part of the solids are decomposed, or the reaction is driven too far resulting in valuable intermediate components being further decomposed into carbon dioxide, which is undesirable if value-adding products are to be obtained.

There still exists a need to find new ways to increase the degradation of organic matter and to achieve a high output of high value end products in a resource effective and thus economically favourable way.

### SUMMARY OF THE INVENTION

An object of the present invention is to provide an efficient process which enables effective utilization of organic matter. The present invention provides a process for fast degradation of organic materials and fermentation of the obtained degraded materials. The initial degradation process is a liquefaction wherein organic materials are degraded into monomers and/or oligomers, and if the organic materials comprise lignocellulosic material, also lignins, are obtained. These short chain monomers and/or oligomers and optionally lignins are fermented to obtain methane as a value-adding end product.

The present invention relates to a method of treating organic materials to produce methane gas, comprising the steps of:
a) subjecting an organic feedstock comprising organic materials to a liquefaction process at subcritical conditions in at least one reaction stage, to obtain a mixture containing low molecular weight materials and optionally lignins;
b) subjecting the obtained mixture containing low molecular weight materials and optionally lignins, to a methane fermentation process;
wherein said organic feedstock comprises liquid water and/or is combined with liquid water before and/or during said liquefaction, and the subcritical conditions for said at least one or more reaction stage(s) in a) is a temperature of 280-374° C. during a reaction time of less than 1 minute.

### DETAILED DESCRIPTION OF THE INVENTION

The term subcritical water refers to liquid water at temperatures between the atmospheric boiling point and the critical temperature (374° C.) of water. By treating an organic substrate, a feed stock, for a short period of time, i.e. below 1 minute, at subcritical conditions, wherein the temperature is in the range of 280-374° C., a monomer and/or oligomer mixture of sugars is created in the liquid phase. Preferably, the obtained liquid phase comprises mixture of monomers and/or oligomers. The solid residue, remaining from lignocellulosic material after the liquefaction, contains mainly lignins and trace amounts of other compounds. The organic substrate may be added to or added water or a water containing phase, and thereafter heated to the subcritical conditions according to the present invention. The organic substrate may also be added hot compressed water, and thus by this addition obtain said subcritical conditions.

Monomers in lignin are connected by different types of ether and carbon-carbon bonds, which are randomly distributed. Lignin also forms numerous bonds to polysaccharides, and in particular hemicelluloses. Due to these crosslinkages lignin containing materials are associated with reduced digestibility. The liquefaction process according to the present invention alters the structure of the material; it opens up the structure of the organic material making the organic material more accessible for different components and conditions. If the organic material contains lignocellulosic materials, the liquefaction breaks up or opens up the lignocellulosic structure making celluloses and hemicelluloses easily accessible to degradation into sugars of lower carbon atom content. Further the lignins present in the lignocellulosic materials are during the liquefaction getting less tightly bonded to polysaccarides and obtains a more open and untangled structure. This more open and untangled structure of the lignin may also make it possible to later degrade the lignin itself since it is by the liquefaction process made more easily available for a subsequent fermentation. Normally lignins are separated from materials to be fermented but according to the present invention the lignins may be present during the fermentation and thus may contribute in the further degradation resulting in an increased amount of obtained value-adding products.

At a temperature of 280-374° C. the structure of the lignin gets untangled and in such a state it is susceptible to methane fermentation. The present invention may be able to degrade not only celluloses and hemicelluloses of lignocellulosic materials but also lignin into a value-adding product, i.e. methane. Thus, the overall conversion of the organic feedstock to methane is increased without a need for separation of the lignin for separate treatments.

By usage of liquefaction at subcritical conditions, before a subsequent fermentation, the original organic materials, the feed stock, are made more easily accessible for digestion and the original organic materials may be chosen from a wide range of substrates. Also, the retention times for the subsequent methane fermentation can be drastically reduced by initially using a liquefaction and thus the process according to the present invention reduces the overall process time. Further, the output of methane or other value adding products can be increased by using the process according to the present invention. If sludge is used as incoming organic material, the sludge will be sanitized and bacteria eliminated, and even viruses may be eliminated. The liquefaction present conditions for optimization of energy and water balances. The residues remaining after the methane fermentation step are reduced due to the combination with a prior liquefaction process compared to conventional methods. The usage of a liquefaction before a methane fermentation step may decrease the needed amounts of enzymes, acids and/or coagulants during the methane fermentation. Another positive feature of using a liquefaction process before a methane fermentation process is that organic materials containing inhibitors, such as inhibiting heavy metals, e.g. cadmium, may with the aid of the liquefaction work better for the bacteria in the methane fermentation step compared to without such a pretreatment.

The degradation of the feedstock in the liquefaction process may be performed without adding chemicals to the processing feedstock. After the degradation of the cellulose and hemicellulose, remaining lignins are either kept in the processing stream and may be degraded in the following methane fermentation step, or are separated from the processing stream. If separated, the lignins could then be processed further to be used as fuel or as chemicals. If the lignins are kept in the processing stream the potential degradation in the following methane fermentation step may increase the overall output of value adding products, e.g. methane, for the process of present invention without extra treatments needed to be done.

Not only the liquid phase obtained after the liquefaction process, may be transferred to a subsequent methane fermentation step to produce methane. Lignins in the slurry have been made more easily accessible by the liquefaction and may be degraded in the subsequent methane fermentation step and in such case contribute to an increased amount of value-adding product, methane, thus would also mean a decreased total residue amount for the overall process.

The liquefaction process may be performed as one single stage or in several subsequent stages. If more than one stage is performed, the obtained liquid phase may be separated from the organic material residue of that stage, and thereafter said organic material residue may be subjected to further liquefaction stages, preferably with separation of the liquid phase after each stage. Also, if more than one stage is used the conditions in the different liquefaction stages may differ. The stages may present different temperatures or temperature profiles, e.g. the liquefaction process starts with a stage at a lower temperature and thereafter each stage have an increased temperature compared to the stage before. The temperature during the one or more liquefaction stages according to the present invention is about 280 to 374° C., preferably 290-370° C., e.g. 290-330° C. For some materials the temperature is preferably 300-360° C., more preferably 300-350° C., such as 310-340° C., 320-340° C. or 330-350° C. The temperature of the process may be increased quickly or slowly but in any case the temperature must reach a temperature of 280 to 374° C. to assure liquefaction according to the present invention. Generally, the temperature in the liquefaction process depends on the incoming organic material. The harder the material is the higher the temperature should be. The temperature for each subsequent stage may be increased compared to the preceding stage or kept constant at a certain temperature. There may be a temperature gradient in the overall liquefaction process that is optimized for breaking the organic components down to suitable oligo- and/or monomers.

If the obtained liquid phase is to be separated from the organic material residue of a liquefaction stage, the temperature is immediately after the liquefaction decreased to at most 200° C. for the separation. Preferably the temperature during separation is in the range 160-200° C., more preferably 160-180° C., which temperature is dependent on that further decomposition during the separation should be suppressed. Moreover, a temperature of at most 200° C. is a level which can be handled today by existing separation equipment, without too much stress being put on the equipment. Examples of suitable separation equipments are centrifuges and hydrocyclones.

If there is more than one reaction stage during the liquefaction every stage should be performed at an increased temperature compared to the previous stage. After each reaction stage the temperature should be decreased to at most 200° C. to stop the ongoing reactions.

Optionally, before the above mentioned liquefaction at a temperature of at least 280° C. is performed the organic feedstock may be subjected to a pretreatment step at a lower temperature of about 230-280° C., preferably 230-270° C. or 240-260° C. Such a pretreatment step may be performed at said temperature for a time period of between 1 second and 2 minutes, e.g. 5 seconds to 1 minute or 10-30 seconds. If a pretreatment step is performed before said liquefaction at 280-374° C. any obtained liquid phase from the pretreatment step must be separated from the organic material residue before the liquefaction at 280-374° C. is performed. Any obtained liquid phase from the pretreatment step may proceed to the methane fermentation step for a further production of value adding products.

The process may involve an iterative liquefaction at sub-critical temperature of an organic feedstock by treatment in hot compressed water (HCW), said process comprising:
feeding an organic feedstock into a reactor no 1 in which part of the feedstock is liquefied;
separating a liquid phase solution no 1, and hence water and water soluble components, from the treated feedstock slurry being discharged from said reactor no 1;
feeding the treated feedstock slurry containing the solid material into a reactor no 2 in which part of the remaining organic materials is liquefied; and
separating a liquid phase solution no 2, and hence water and water soluble components, from further treated feedstock slurry being discharged from said reactor no 2.
Additional and subsequent reactor(s) and hence feeding and separating steps are involved in the process so that liquid phase solutions no 3 to N are separated after respective reactor no 3 to N. The number of reactors may vary according to the present invention, depending on the feedstock and desired composition on separated products.

The usage of water at sub-critical conditions is chosen since it is considered better from an energy point of view, and that corrosion is lower on the apparatus and the risk of pushing the reaction too far obtaining water and carbon dioxide is lower compared to usage of water at supercritical conditions.

The reaction time is an important feature of the present invention. If the reaction time is set too short, the conversion is not made enough to obtain a high yield of desirable monomers and/or oligomers, and if the reaction time is set too long, too high percentage of the monomers have further degraded into carbon dioxide and water, i.e. so called continued detrimental decomposition has resulted. The reaction time in said one or more reaction stage(s) in the liquefaction process is less than one minute, preferably 0.05 to 55 seconds, preferably 0.5 to 50 seconds, preferably 1 to 40 seconds, preferably 5 to 40 seconds, and most preferably 10 to 30 seconds.

The stages of the liquefaction process may be performed in terms of batchwise, semi-batchwise or continuous process. A continuous process is preferred. The reactors used could be batch reactors, alone or in series, or flow reactors, such as tubular reactors. Optionally several flow reactors can be used, for instance two reactors out of sync, where loading of biomass is performed in one reactor while the reaction is performed in a second reactor, thus is enabling a continuous net flow. If a flow reactor is used, a slurry of organic materials is pumped at high pressure through a heating region where it is exposed to temperatures that bring the water to sub-critical conditions. Preferably, the residence time of the slurry in the heating region at the previously disclosed sub-critical conditions is the same as the reaction time mentioned above.

The sub-critical conditions required for the liquefaction are obtained by heating and optionally pressurizing a mixture of organic substances and a water containing liquid, to the required temperature, and/or organic substances are subjected to hot compressed water to reach the required temperature. In one embodiment of the present invention a slurry of organic material and liquid water is heated and pressurized until the sub-critical conditions according to the present invention have been reached. In another embodiment organic material is mixed with pressurized liquid water and then heated until the sub-critical conditions according to the present invention have been reached. In yet another embodiment of the present invention organic material is mixed with pressurized liquid water and then heated, thereafter addition of hot compressed water is made until the sub-critical conditions according to the present invention have been reached. Still another embodiment relates to organic material being mixed with pressurized liquid water and then heated; thereafter addition of hot compressed water is made until the sub-critical conditions according to the present invention have been reached.

HCW is injected into a batch reactor by one cycle or repeated cycles; a series of batch reactors by one cycle or by repeated cycles; or a flow reactor by one cycle.

When liquefaction of a feedstock is performed in only one reactor, the reaction may not driven far enough so that only part of the solids are liquefied, or valuable components are further decomposed, which is undesirable, when the reaction is driven too far. Therefore, it is of interest to perform the liquefaction in iterative steps and separating the valuable fractions after each reactor before going to next loop when liquefying the remaining solids. By doing so, it is according to the present invention possible to optimize each reaction step differently and more economically beneficial in comparison to trying to liquefy in only one or possibly two steps. By using several liquefaction steps, the obtained specific fractions may be optimized to produce specific degraded compounds that may be considered value-adding products and may be further used in other processes or applications. By use of several reactors in the process according to the present invention, it is possible to optimise the refining of an organic slurry feedstock and hence the separation of components from the feedstock. By use of several reactor steps, it is possible to involve different heating and cooling steps and temperature ranges during the process according to the present invention. This is done in order to optimize the entire fractionating of the feedstock and to minimize the level of undesired decomposition products. Moreover, the pressure also changes during the process, either naturally during the temperature increase and decrease or actively in or between different reactors as a process driving parameter.

Methane fermentation is capable of converting almost all types of polymeric materials to methane and carbon dioxide under anaerobic conditions. This is achieved as a result of the consecutive biochemical breakdown of polymers to methane and carbon dioxide in an environment in which varieties of microorganisms which include fermentative microbes (acidogens); hydrogen-producing, acetate-forming microbes (acetogens); and methane-producing microbes (methanogens) harmoniously grow and produce reduced end-products. The methane fermentation in the present invention is not particularly limited and can be carried out by applying a conventional method as appropriate.

As an example, the obtained mixture of low molecular weight substances, optionally lignins, and fermenting methane producing micro-organisms, e.g. bacteria, is fed into a methane fermentation reactor. The methane fermentation reactor is kept at a predetermined temperature, and methane fermentation is carried out for a predetermined retention time while the contents of the reactor are stirred appropriately. The generated methane gas is collected in a conventional manner. The methane fermentation may be either a batch type methane fermentation or continuous type methane fermentation.

As microorganisms for use in the methane fermentation, conventionally known methanogens or the like can be used. In order to optimize the methane output the bacteria should be adapted to favor the methane forming bacteria. However, other bacteria could also be favored if focus more lies on reducing the amount of waste residue left after the methane fermentation step. It is desirable to reduce the waste volumes as much as possible.

The temperature in the methane fermentation reactor may be set to conventionally known temperatures suitable for microorganisms for use in methane fermentation. Mesophilic digestion is performed at temperatures between 20° C. and 40° C., typically about 35-37° C. Thermophilic digestion is performed at temperatures above 50° C., e.g. 50-70° C. If the feed stock in the treatment of the present invention is a waste material, for example like sludge, an application onto farmlands after treatment may be desirable and thus would make a methane fermentation at a higher temperature of about 50-55° C. preferable in view of the sludge getting sanitized.

The methane fermentation is carried out under conventional temperature conditions and due to the liquefaction preceding the methane fermentation, the retention times may be decreased considerably. If only liquid phase is methane fermented the retention times are lower compared to if solids, like e.g. lignins, are present. Examples of retention times for the methane fermentation are 10-20 days, or 10-48 hours.

By addition of small amounts of iron coagulants and/or trace amounts of heavy metals like e.g. cobalt, the amount of methane produced during methane fermentation is increased. Such compounds may be added to the feedstock before the liquefaction of the present invention and the overall degradation of the initial organic matter may be increased further. Iron coagulants and/or trace elements of heavy metals may be added to the organic material before, during and/or after the liquefaction process.

The organic materials used as feed stock in the process according to the present invention are various vegetations and wastes. The vegetation may be annual or perennial. Examples of annual plants are corn, lettuce, pea, cauliflower, bean and hemp. Preferably lignocellulosic biomass or waste containing polymers are used, e.g. materials including starch, cellulose, hemicellulose, lignin, lignocellulose or a combination thereof. Examples of suitable materials are wastes from agriculture, sewage treatments, slaughterhouses, food industry, restaurants and households; plastics; cardboard; paper; manure; corn; rice; rice husk; wood; stumps; roots; straw; hemp; salix; reed; nutshells; sugar cane; bagasse; grass; sugar beet; wheat; barley; rye; oats; potato; tapioca; rice; and algae.

1. Method of treating organic materials to produce methane gas, comprising:
a) subjecting an organic feedstock comprising organic materials to a liquefaction process at subcritical conditions in at least one reaction stage, to obtain a mixture containing low molecular weight materials and optionally lignins;
b) subjecting the obtained mixture containing low molecular weight materials and optionally lignins, to a methane fermentation process;
wherein said organic feedstock comprises liquid water and/or is combined with liquid water before and/or during said liquefaction, and the subcritical conditions for said at least one or more reaction stage(s) in a) is a temperature of 280-374° C. during a reaction time of less than 1 minute, and
wherein if more than one reaction stage is used in step a) the obtained mixture after each reaction stage is subjected to a separation of the produced low molecular weight materials from the remaining solid materials of the treated feedstock.

2. Method according to claim 1, wherein the reaction time in said one or more reaction stage(s) in the liquefaction process is 0.05 to 55 seconds, preferably 0.5 to 50 seconds, preferably 1 to 40 seconds, preferably 5 to 40 seconds, and most preferably 10 to 30 seconds.

3. Method according to claim 1, wherein the reaction temperature in said one or more reaction stage(s) in the liquefaction process is between 290 and 370° C., preferably 300-360° C., and more preferably 300-350° C.

4. Method according to claim 1, wherein said organic feedstock is combined with liquid water before and/or during said liquefaction by addition of hot compressed liquid water.

5. Method according to claim 1, wherein said separation is made at a temperature of at most 200° C.

6. Method according to claim 1, wherein if more than one reaction stage is used in step a) the temperature in each reaction stage is the same or increasing for each subsequent stage.

7. Method according to claim 1, wherein the organic feedstock is selected from vegetations and wastes.

8. Method according to claim 7, wherein the organic feedstock is lignocellulosic biomass or waste comprising polymers.

9. Method according to claim 7, wherein the organic feedstock is chosen from wastes from agriculture, sewage treatments, slaughterhouses, food industry, restaurants and households; plastics; cardboard; paper; manure; corn; rice; rice husk; wood; stumps; roots; straw; hemp; salix; reed; nutshells; sugar cane; bagasse; grass; sugar beet; wheat; barley; rye; oats; potato; tapioca; rice; and algae; or any combination thereof.

10. Method according to claim 1, wherein the liquefaction process is performed in at least one batch reactor or at least one flow reactor.

11. Method according to claim 10, wherein the hot compressed water is injected into a batch reactor by one cycle or repeated cycles; a series of batch reactors by one cycle or by repeated cycles; or a flow reactor by one cycle.

12. Method according to claim 1, wherein the liquefaction process at temperature of 280-374° C. is preceded by a pretreatment step at a temperature of about 230-280° C. for a time period of between 1 second and 2 minutes.

13. Method according to claim 4, wherein the hot compressed water is injected into a batch reactor by one cycle or repeated cycles; a series of batch reactors by one cycle or by repeated cycles; or a flow reactor by one cycle.
